(19) United States
(12) Patent Application Publication
(10) Pub. No.: US 2010/0028938 A1
(43) Pub. Date: Feb. 4, 2010

(54) METHOD OF SCREEING FOR PATHOGENS

(76) Inventors: Alistair J. Brown, Hampshire (GB); Stephen Dimmer, Hampshire (GB)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/533,083

(22) Filed: Jul. 31, 2009

(30) Foreign Application Priority Data
Aug. 1, 2008 (DE) GB0814099.8

(51) Int. Cl. C12Q 1/04

(52) U.S. Cl. 435/34

(57) ABSTRACT

Disclosed is a method of screening for the presence of MRSA in a subject, the method comprising the steps of:
(a) obtaining a suitable sample from the subject using a swab or other suitable sample collection device;
(b) substantially immediately after performing step (a), contacting the swab or other sample collection device with an identification medium, said medium comprising:
   (i) nutrients to allow the growth of MRSA;
   (ii) at least one inhibitor substance to inhibit preferentially the growth of organisms other than MRSA; and
   (iii) a visual indicator substance which generates a visual indication of the growth of MRSA if present;
(c) incubating the identification medium, typically whilst still in contact with the swab or other sample collection device, in conditions which are sufficient to cause the growth of MRSA; and, after sufficient time has elapsed;
(d) inspecting the medium to determine the presence or absence of the visual indication of the growth of MRSA.

### CROSS-REFERENCES TO RELATED APPLICATION

This application claims priority based on German Patent Application No. GB 0814099.8, filed Aug. 1, 2008, of which is hereby incorporated by reference in its entirety.

### FIELD OF THE INVENTION

The invention relates to a method of screening for pathogens, in particular to a rapid method of screening for the presence of meticillin-resistant Staphylococcus aureus (MRSA), and to media and kits of use in such methods.

### BACKGROUND OF THE INVENTION

Nosocomial or iatrogenic infection by MRSA is a well-known and widespread problem in the UK and elsewhere. In an effort to help control the incidence of MRSA infections in the UK, it is planned to screen every patient entering hospital, in order to identify those individuals who may be asymptomatic carriers. It is estimated that between 10 and 20% of the general population in the UK may be carriers of MRSA.

Currently, conventional methods of screening entail taking a swab (such as a nasal swab) from an individual at the point of care. The swab is then placed in a sterile container and forwarded to the microbiology lab in the hospital, where it is unpackaged and used to inoculate a screening plate. The plate contains a solid medium which has ingredients to stimulate the growth of any MRSA bacteria present, but also contains some sort of marker which gives a visible indication if MRSA are present.

One of the best screening media currently available is that sold under the trade name “Brilliance” MRSA Agar, available from Oxoid Limited. This is an opaque medium incorporating a chomogen that yields a blue colour as a result of phosphatase activity. The enzyme phosphatase is present in many staphylococci, including S. aureus. Accordingly, to allow the medium to differentiate MRSA from other staphylococci, the medium also comprises a combination of antibacterial compounds selected to inhibit the growth of a wide variety of likely competitor organisims, including meticillin-sensitive Staph. aureus (MSSA). Also incorporated in the medium are compounds to suppress the expression of phosphatase activity in other staphylococci, thus providing a high level of sensitivity and specificity. MRSA, if present, are discernible by their distinctive bright blue colouration on the medium.

Although the current method and medium are satisfactory, it is still necessary to transfer the sample from the collection swab to a growth medium and to incubate the plates at 37° C. for at least 18 hours after inoculation with the swabs. The transfer step consumes the time of a laboratory technician, especially if screening is to be implemented on a large scale, and increases the risk of contamination or other mishap.

Rambach et al (US2006/0035309) discloses a method for detecting meticillin-resistant micro-organisms using a solid medium comprising a cephalosporin and a chromogenic agent which releases a chromophore after hydrolysis by an enzyme which is produced by the micro-organisms to be detected. Rambach et al do not disclose the use of liquid or semi-solid media and do not disclose the use of an appropriate medium at the point-of-care.

Given the very large numbers of individuals whom it is now envisaged will need to be screened, the average hospital laboratory will need to use many thousands of plates a year, and at any one time there are likely to be several hundred plates requiring incubation or checking which will take up a lot of volume in incubators or on desks.

Accordingly, there is an urgent need for a method of screening for MRSA which eliminates the labour-intensive transfer of samples from collection swab to growth medium (along with the risks associated with such transfer), is cost-effective even if performed on a large scale and uses less space.

### SUMMARY OF THE INVENTION

The invention also thus provides a method of screening for the presence of MRSA in a subject, the method comprising the steps of:
(a) obtaining a suitable sample from the subject using a swab or other suitable sample collection device;
(b) substantially immediately after performing step (a), contacting the swab or other sample collection device with an identification medium, said medium comprising:
   (i) nutrients to allow the growth of MRSA;
   (ii) at least one inhibitor substance to inhibit preferentially the growth of organisms other than MRSA; and
   (iii) a visual indicator substance which generates a visual indication of the growth of MRSA if present;
(c) incubating the identification medium, typically whilst still in contact with the swab or other sample collection device, in conditions which are sufficient to cause the growth of MRSA; and, after sufficient time has elapsed;
(d) inspecting the medium to determine the presence or absence of the visual indication of the growth of MRSA; wherein both steps (a) and (b) take place at the point of care (e.g. typically in the presence of the subject).

The step (b) takes place substantially immediately after step (a). That is, in practice, it is desirable that both steps (a) and (b) take place at the point of care, with the health worker swabbing the subject and then substantially immediately contacting the swab or other sample collection device with the identification medium. “Substantially immediately” in this context means that after obtaining a sample from the subject, the swab or other sample collecting device is contacted with the identification medium typically within 3 minutes, preferably within 2 minutes and most preferably within 1 minute.

The invention also thus provides a method of screening for the presence of MRSA in a subject, the method comprising the steps of:
(a) obtaining a suitable sample from the subject using a swab or other suitable sample collection device;
(b) contacting the swab or other sample collection device with an identification medium, said medium comprising:
   (i) nutrients to allow the growth of MRSA;
   (ii) at least one inhibitor substance to inhibit preferentially the growth of organisms other than MRSA; and
   (iii) a visual indicator substance which generates a visual indication of the growth of MRSA if present;
(c) incubating the identification medium, typically whilst still in contact with the swab or other sample collection device, in conditions which are sufficient to cause the growth of MRSA; and, after sufficient time has elapsed;
(d) inspecting the medium to determine the presence or absence of the visual indication of the growth of MRSA; wherein both steps (a) and (b) take place at the point of care (e.g. typically in the presence of the subject).

The swab sample/medium combination will then typically be passed to a microbiology lab, where incubation step (c) and inspection step (d) will normally take place.

It is preferred that, once the swab has been contacted with the medium (step b), the swab is maintained in contact with the medium during at least part, and preferably all, of step (c), although if desired for some reason, the swab or other sample collection device can be removed from contact with the medium after a suitable time has elapsed. Further, once step (b) has occurred, it is preferred that the medium is maintained at a temperature in the range 16-40° C., preferably 16-38° C., as soon as is practicable after the swab is contacted with the medium, and that step (c) should follow as soon as is practicable after performing step (b).

In a second aspect, the invention provides a kit for use in the methods of the first aspect, the kit comprising: a sterile package containing a swab or other sample collection device, and an identification medium as defined in the first aspect above.

The swab of use in the various aspects of the invention may be of substantially conventional design, and is readily available commercially from many sources. The swab may be, for example, tipped with cotton, rayon, nylon, Dacron™, or flocked.

Advantageously, the swab is provided as an integral, but removable, component of a container used to contain the identification medium. In use, the swab component is removed from the container, contacted with the relevant portion of the subject to be swabbed, and then reintroduced into the container in such a way that the swab contacts the medium therein. The container will typically comprise a tube, with a volume in the range 3-30 mls, preferably 5-25 mls, more preferably 10-25 mls, which can be accommodated in conventional laboratory racking for test tubes.

The identification medium of use in the invention will comprise nutrients sufficient for the growth of S. aureus, such as peptones, yeast extract, various salts and minerals, and a source of carbohydrate, such as glucose, dextrin or starch. The medium is preferably liquid, or semi-solid, but could be solidified. For present purposes, a semi-solid medium is one which is quasi-solid (i.e. can support its own weight and hold its shape, but has the ability to flow under pressure and will conform to the shape of a surface which applies pressure to it). A quasi-solid medium can be obtained, for example, by utilising a concentration of gelling agent (e.g. agar, agarose, Sephadex™ or gelatine) lower than that conventionally used to prepare solid media. As an illustration, a concentration of agar in the range 1-8 grams per litre, preferably 2-7 grams per litre, most preferably 3-6 grams per litre, may be used to provide a quasi-solid medium.

Liquid and quasi-solid media are preferred since, inter alia, they allow for greater contact of the medium with the swab than does a solid medium.

The medium will further comprise at least one inhibitor substance to inhibit the growth of competitor organisms (i.e. those organisms other than MRSA). In practice, the medium is likely to comprise a plurality of inhibitor substances. Such a plurality of inhibitor substances will normally be required because of the variety of competitor organisms that may be present in the sample, which may include, for example, coagulase negative staphylococci (CoNS), Gram negative organisms, Enterococcus spp., and yeasts. Accordingly the medium will typically comprise one or more, preferably two or more, and most preferably three or more of the following inhibitors: polymyxin B, colistin sulphate, aztreonam, lithium chloride, deferoxamine mesylate, and amphotericin B, Fluconazole or another antifungal compound.

In addition, because of the need to inhibit, in particular, the growth of MSSA, the medium will preferably comprise one or more of the following; meticillin; oxacillin, a cephalosporin or a cephamycin.

The inhibitor substances are selected, and are present at a concentration, such that they preferentially inhibit competitor organisms. That is, they inhibit the growth of competitor organisms to a greater extent than they inhibit the growth of MRSA.

Further, the identification medium will comprise at least one visual indicator substance, which generates, directly or indirectly, a visual signal indicative of the growth of MRSA. Typically, but not necessarily, the visual signal will take the form of a colour change. This could be, for example, a change from one colour to a different colour; a change from colourless to a colour, or vice versa. In one embodiment, the indicator substance may comprise an indicator of pH change or redox change, such changes occurring as a result of the fermentation or alteration of a substrate within the medium, due to the action of one or more enzymes elaborated by MRSA. Examples of such indicators include, for example, phenol red, neutral red or aniline blue.

In a preferred embodiment however, the indicator substance comprises a chromogenic or fluorogenic compound. Preferably the indicator substance is altered by the action of a phosphatase and/or a ribosidase produced by MRSA. Preferred chromogens are altered by the action of MRSA phosphatase to produce a coloured product. Particularly preferred chromogens for inclusion in the medium include indoxyl phosphates. Preferred fluorogens include umbelliferyl phosphate fluorogens, such as 4-methylumbelliferyl phosphate.

In some embodiments, the medium may comprise a pH indicator as the “visual indicator substance”, or may comprise a fluorogen “visual indicator substance”, but not both a pH indicator and a fluorogen, because use of two indicators does not significantly increase the ease or sensitivity of detection, but rather adds to the cost and complexity of making the medium.

Finally, the medium may comprise a gelling agent such as agar, Sephadex™, agarose or gelatine, to provide a semi-solid medium, thus ensuring the swab or other sample collection device remains in contact with the medium.

The person skilled in the art will, with the benefit of the present disclosure, be able to determine appropriate concentrations for the various ingredients of the medium, and sample formulations are given in the examples below.

The method of the invention involves obtaining a suitable sample from the subject. This will generally entail swabbing the nostrils and/or the hands of the subject, which are those sites most commonly colonised by MRSA, although other portions of the subjects's skin may also or alternatively be swabbed.

The method is conveniently performed using a kit in accordance with the second aspect of the invention. In one embodiment, the kit is provided as a sterile package, with an impermeable wrapping such as a metalised foil or a synthetic plastics film or bag, within which is provided a glass or synthetic plastics tube, bottle or other container housing an aliquot of the growth/identification medium, sealed with a removable sealing means, such as a screw cap or other device. The kit will also comprise a label to accommodate relevant information, such as the subject's name, the date of sampling, the site on the subject sampled, and so on. The label may be provided separately or may be provided already attached to the tube, bottle or container housing the medium. Desirably a self adhesive label is used.

The package will also comprise one or more swabs such as a wooden or plastics—handled swab, or other suitable sample collection device. These will be sterile (e.g. uv-irradiated) and may be packaged separately from the container housing the medium. In an alternative embodiment, a swab is provided as an integral part of the container. As an example, the container may take the form of a tube with a screw cap. Projecting upwards from the cap is an integrally formed swab. To obtain a sample, the cap is unscrewed from the tube, and the swab contacted with the subject. The cap is then replaced in the tube in the reverse orientation, with the swab projecting downwards into the medium. If desired the cap can be provided with a screw-threaded portion in an opposite sense, to assist in replacing the cap on the tube in the reverse orientation. Alternatively, when inserted into the container in the reverse orientation the cap may form a seal by means of a stopper or bung effect.

Once contacted with the medium, the swab could in principle be removed after agitation. However, it is preferable to retain the swab in contact with the medium during the incubation step to ensure that at least some of the MRSA bacteria present are transferred to the medium.

The incubation conditions will be apparent to those skilled in the art and will usually involve incubation at a temperature above ambient, preferably in the range 30-40° C., and most preferably at about 37° C. The length of time required in order to produce a visual signal indicative of the presence of MRSA will vary depending on the precise conditions of the assay, the composition of the medium, and the physiological state of the organisms at the time of sampling (e.g. whether or not physiologically stressed and to what extent). Typically an incubation of about 18-24 hours is appropriate, but in some conditions the inventors believe it may be possible to obtain a presumptive result after as little as 6 hours' incubation in step (c).

The medium will most conveniently be directly inspected by a human observer, but if desired an automated system could be employed, the medium being interrogated by a machine such as a spectrophotometer or other colour or fluorescence-sensitive device.

The method of the invention offers four principle advantages over conventional prior art methods.

Firstly, in the prior art, a swab sample is taken from a patient and then transferred to the hospital lab either in a sterile container without medium or in a sterile transfer medium (such as saline) which does not promote the growth of MRSA. Once the swab reaches the laboratory, no immediate action may be taken: the swab may be held on a bench and batched until a sufficient number have been received in order to justify a technician's time in using the swabs to inoculate plates.

In contrast, with the method of the invention, the swab is immediately placed in contact with a medium which promotes and stimulates the growth of MRSA, whilst simultaneously preferentially inhibiting the growth of competitor organisms. This gives the MRSA organisms a “head start” relative to the prior art method, such that results can be obtained more rapidly after swabbing of the subject has taken place.

Secondly, no manipulation of the samples is required when received in the laboratory, and they are therefore less likely to be delayed before processing. Instead, a technician simply has to place the sample tube or container in an incubator. Reducing the number of manipulations of the samples also reduces the risk of contamination.

Thirdly, the tubes or containers employed in the present invention occupy far less volume than conventional plates, thus saving on desktop space and incubator space, which is very significant, when large numbers of samples are involved. Petri dishes, used for making plates of solid culture media, are usually 90-150 mm in diameter, whereas the tubes or containers for use in the present method are typically only 10-20 mm in diameter.

Fourthly, the method of the present invention should provide greater sensitivity than conventional methods. Where a swab is taken and sent to the laboratory in isolation (i.e. not in contact with a liquid or semi-solid medium), the organisms present on the swab will be in sub-optimal conditions. Some may die, and even if the organisms do not die they will become physiologically stressed, which may mean they are outgrown by more robust competitor organisms when the swab is used to inoculate a solid growth medium, leading to a false negative result.

Even if the swab is inserted into a conventional transport medium (such as Amies medium), the conditions will still be sub-optimal. By way of explanation, transport media are intended to hold microorganisms in a state of relative ‘stasis’, such that the number of viable cells subsequently recovered reflects that contained within the specimen at time of collection. This means that any delay between sampling and inoculation of a growth medium does not cause an increase in numbers of the target organisms. Sensitivity of the final growth medium is dependent on the number of viable cells remaining in the transport swab at the point of inoculation.

In contrast, the method of the invention ensures that the number of organisms in the specimen start to increase as soon as it is placed into the differential MRSA transport medium, provided that the medium remains at a temperature between 16-40° C., preferably 16-38° C. As the target organisms are provided with the appropriate nutrients, the number of viable cells is likely to increase. Similarly, non-target organisms will be inhibited as soon as the specimen is placed into the MRSA transport medium, reducing the likelihood of over-growth of ‘background flora’ and resulting competitive inhibition of MRSA. This increases sensitivity of the method.

Finally, the method of the invention is labour saving. The current practices in microbiology laboratories involve inoculation of a screening swab (usually nasal swab taken from a patient and contained in a transport medium) onto a solid or liquid growth medium. The growth medium is then incubated to stimulate growth of MRSA. This practice is carried out by laboratory staff. Many swabs are received by laboratories each day for MRSA screening, making this a labour-and time-intensive procedure.

In a third aspect the invention provides an identification medium suitable for the screening of samples from a subject for the presence of MRSA, the medium being liquid or semi-solid and comprising:
(i) nutrients to allow the growth of MRSA;
(ii) at least one inhibitor substance to inhibit preferentially the growth of organisms other than MRSA; and
(iii) a visual indicator substance which generates a visual indication of the growth of MRSA if present.

The medium is preferably initially colourless and translucent, and becomes coloured if MRSA organisms grow during incubation of the medium. Other preferred features of the medium are as recited above in the context of the first and second aspects of the invention.

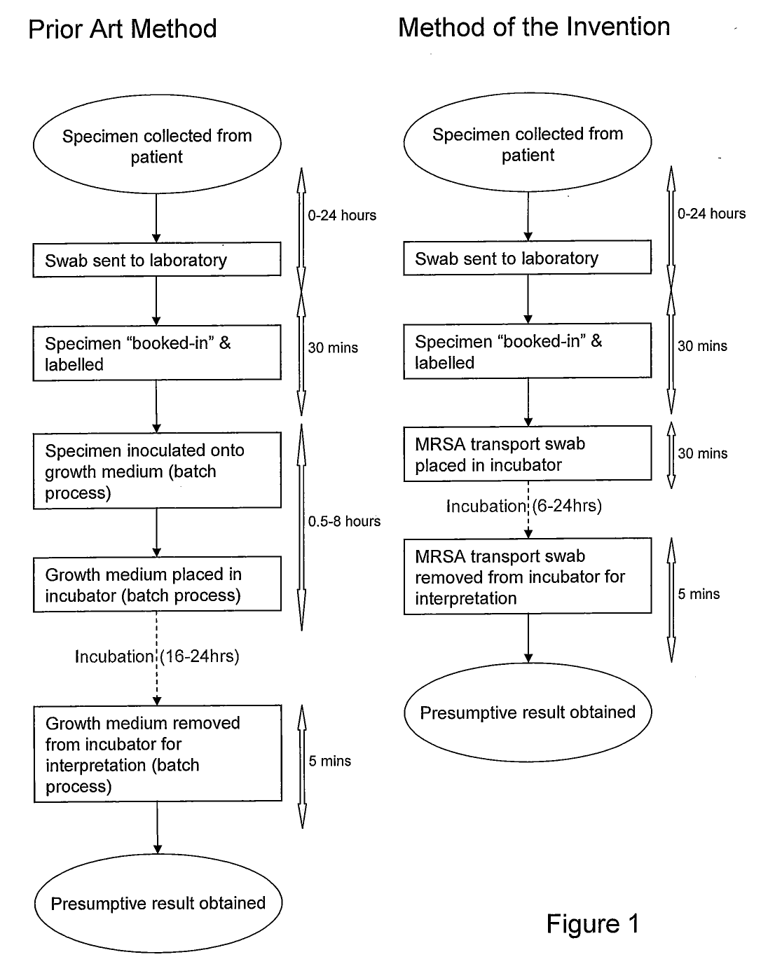

The invention will now be further described by way of illustrative example, and with reference to the accompanying drawing FIG. 1, which is a flow chart comparing the prior art method with the method of the invention.

### EXAMPLE 1

### A Liquid (Broth) Medium Formulation Suitable for Use in the Various Aspects of the Invention

The liquid medium will contain the following:

a nutritive broth base, such as tryptone soy broth, nutrient broth;

at least one substance to inhibit the growth of organisms other than MRSA, such as meticillin, oxacillin, various cephalosporins or cephamycins;

at least one substance to inhibit the growth of organisms other than Staphylococcus aureus. Such substances include polymixin B, colistin sulphate, aztreonam, lithium chloride, deferoxamine mesylate, amphotericin B, fluconazol, anisomycin or other antifungal compounds; and

at least one indicator compound to differentiate the growth of MRSA from other organisms. This can be an indicator of pH or redox change resulting from fermentation of carbohydrate contained within said medium. Such indicators could include phenol red, neutral red, bromocresol purple, bromothymol blue, or aniline blue.

In another example, the indicator can be substituted for at least one chromogenic compound to detect enzyme activity specific to Staphylococcus aureus. Examples of this are indoxyl phosphate chromogens which detect phosphatase activity.

In another formulation, the indicator can be substituted for at least one fluorogenic compound to detect enzyme activity specific to Staphylococcus aureus. An example of this is the 4-methylumbelliferyl phosphate fluorogen which detects phosphatase activity.

One sample formulation is as follows:

Typical composition per litre of medium:

| | | |
|---|---|---|
| Peptone | 10-15 | g |
| Beef Extract | 1-5 | g |
| Mannitol | 5-15 | g |
| Sodium chloride | 50-75 | g |
| Phenol Red | 20-30 | mg |
| Oxacillin | 4-6 | mg |
| Polymixin B | 10-15 | mg |
| Aztreonam | 10-25 | mg |

### EXAMPLE 2

### A Semi-Solid Medium Formulation Suitable for Use in the Various Aspects of the Invention

The semi-solid medium will contain the following:

a nutritive broth base, such as tryptone soy broth or nutrient broth, containing at least one gelling agent. Such gelling agents could include agar, agarose, Sephadex™ or gelatine. A concentration range would be required to provide a gel strength which prevents spillage of the medium from the culture tube or bottle, and enables optimal contact of the medium with the swab. One example is a concentration of agar between 1 and 8 grams per litre;

at least one substance to inhibit the growth of organisms other than MRSA, such as meticillin, oxacillin, various cephalosporins or cephamycins;

at least one substance to inhibit the growth of organisms other than Staphylococcus aureus. Such substances include polymixin B, colistin sulphate, aztreonam, lithium chloride, deferoxamine mesylate, amphotericin B, fluconazole, anisomycin or other antifungal compounds; and

at least one indicator compound to differentiate the growth of MRSA from other organisms. This can be an indicator of pH or redox change resulting from fermentation of carbohydrate contained within said medium. Such indicators could include phenol red, neutral red, bromocresol purple, bromothymol blue, or aniline blue.

One sample formulation is as follows:

Typical composition per litre of medium:

| | | |
|---|---|---|
| Peptone | 10-15 | g |
| Beef Extract | 1-5 | g |
| Mannitol | 5-15 | g |
| Sodium chloride | 50-75 | g |
| Agar | 2-8 | g |
| Phenol Red | 20-30 | mg |
| Oxacillin | 4-6 | mg |
| Polymixin B | 10-15 | mg |
| Aztreonam | 10-25 | mg |

In another example, the indicator can be substituted for at least one chromogenic compound to detect enzyme activity specific to Staphylococcus aureus. Examples of this are indoxyl phosphate chromogens which detect phosphatase activity.

In another example, the indicator can be substituted for at least one fluorogenic compound to detect enzyme activity specific to Staphylococcus aureus. An example of this is the 4-methylumbelliferyl phosphate fluorogen which detects phosphatase activity.

### EXAMPLE 3

As shown in the Flow diagrams in FIG. 1, the method of the invention (timeline on the right hand side) provides a more streamlined process than the prior art method (timeline on the left hand side) and, in practice, gives rise to a presumptive result several hours in advance of the conventional prior art technique.

1. A method of screening for the presence of MRSA in a subject, the method comprising the steps of:
(a) obtaining a sample from the subject using a swab or other sample collection device;
(b) contacting an identification medium with the swab or other sample collection device containing the sample, said medium comprising:
(i) nutrients sufficient to allow the growth of MRSA if present;
(ii) at least one inhibitor substance to inhibit preferentially the growth of organisms other than MRSA; and
(iii) a visual indicator substance which generates a visual indication of the growth of MRSA if present;
(c) incubating the identification medium, after or while still in contact with the swab or other sample collection device, in conditions sufficient to cause the growth of MRSA if present; and
(d) inspecting the medium to determine the presence or absence of the visual indication of the growth of MRSA.

2-21. (canceled)

22. The method according to claim 1, wherein the identification medium is liquid or a semi-so lid.

23. The method according to claim 1, wherein steps (a) and (b) are performed at the point of care of the subject.

24. The method according to claim 1, wherein the medium comprises a source of carbohydrate utilizable as an energy source by MRSA and one or more of the following sources of nutrients: peptones; yeast extract; salts; and minerals.

25. The method according to claim 1, wherein the medium comprises two or more of the following substances at a concentration sufficient preferentially to inhibit the growth of competitor organisms: polymyxin B; colistin sulphate; aztreonam; lithium chloride; deferoxamine mesylate; and amphotericin B, fluconazole or another antifungal compound.

26. The method according to claim 25, wherein the medium comprises three or more of the inhibitor substances recited in claim 5, at a concentration sufficient preferentially to inhibit the growth of competitor organisms.

27. The method according to claim 1, wherein the medium comprises, at a concentration sufficient preferentially to inhibit the growth of MSSA or other competitor organisms if present, one or more of the following: meticillin; oxacillin; a cephalosporin; and a cephamycin.

28. The method according to claim 27, wherein the medium comprises, at a concentration sufficient preferentially to inhibit the growth of MSSA or other competitor organisms, two or more of the antibacterial substances recited in claim 7.

29. The method according to claim 1, wherein the indicator substance comprises a chromogen or a fluorogen.

30. The method according to claim 1, wherein the indicator substance comprises a chromogen or fluorogen substrate for MRSA phosphatase.

31. The method according to claim 1, wherein step (c) comprises incubating the medium, after or while still in contact with the swab or other sample collection device, at a temperature in the range of 35-38° C. for a time period in the range of 16-24 hours.

32. The method according to claim 1, wherein the visual indicator substance generates a signal specifically indicating the growth of MRSA.

33. The method according to claim 1, comprising use of a medium which comprises a pH indicator or a fluorogen, but not both.

34. A kit for use in performing the method of claim 1, the kit comprising a sterile package containing a swab or other sample collection device and an identification medium as recited in claim 1.

35. The kit according to claim 34, wherein the identification medium is provided in liquid or semi-solid form and is contained within a sterile container having a removable and resealable sealing means.

36. The kit according to claim 35, wherein the container comprises a screw cap.

37. The kit according to claim 35, wherein the swab or other sample collection device is provided as an integral part of the container.

38. The kit according to claim 34, further comprising instructions for performing the method of claim 1.

39. The kit according to claim 34, comprising a medium which comprises a pH indicator or a fluorogen, but not both.

40. A liquid or semi-solid identification medium suitable for use in performing the method of claim 1, the medium being as recited in claim 1.

41. The medium according to claim 40, the medium comprising a pH indicator or a fluorogen, but not both.
